(19) United States
(12) Patent Application Publication
(10) Pub. No.: US 2007/0218134 A1
(43) Pub. Date: Sep. 20, 2007

(54) Compositions Comprising Organic Compounds

(76) Inventors: Sabine Desset-Brethes, Prattein (CH); Andreas Meyer, Neuenburg (DE)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 10/580,895

(22) PCT Filed: Nov. 25, 2004
(86) PCT No.: PCT/EP04/13419

§ 371 Date: Mar. 13, 2007

Related U.S. Application Data
(60) Provisional application No. 60/525,373, filed on Nov. 26, 2003.

(51) Int. Cl. A61K 9/22

(52) U.S. Cl. 424/471

(57) ABSTRACT

The present invention relates to pharmaceutical compositions for sustained release comprising as active ingredient an HMG-CoA reductase inhibitor or a pharmaceutically acceptable salt thereof, said composition comprising a core consisting of an inner phase (internal) and an outer phase (external) wherein the outer phase does not comprise a matrix former and wherein the core is first coated with a non functional film coat and then with an enteric coat.

The present invention relates to pharmaceutical compositions for sustained release comprising as active ingredient an HMG-CoA reductase inhibitor or a pharmaceutically acceptable salt thereof, said composition comprising a core consisting of an inner phase (internal) and an outer phase (external) wherein the outer phase does not comprise a matrix former and wherein the core is first coated with a non functional film coat and then with an enteric coat.

When using the composition according to the present invention, unexpected advantages can be demonstrated.

It has been surprisingly found that the composition according to the invention more advantageously increases the distribution of the HMG-CoA reductase inhibitor to the liver due to the slow drug release and decreases the drug plasma levels and consequently the distribution to the muscle tissue due to the slow drug release. The consequence is a better tolerability as compared to the tolerability of the same dose of an immediate release composition of the HMG-CoA reductase inhibitor. Because of the improved tolerability of the extended release composition higher doses can be administered leading to higher efficacy of the drug. The improved tolerability of the pharmaceutical composition and consequently higher efficacy is based on a well adapted extended release profile. An improved adapted extended release profile is due notably to the presence of the matrix former or a mixture of matrix formers of different viscosities in the composition according to the present invention creating an advantageous diffusion barrier by hydrogel formation of the matrix in aqueous media, this matrix core being coated with an enteric film preventing advantageously a fast active release in the stomach.

Furthermore, a small size of the pharmaceutical dosage form and, in parallel, the possibility to apply a low dose formulation of active ingredient induce a better tolerability of the active ingredient.

It was surprisingly found that the compositions according to the invention have improved safety and tolerability than other statin sustained release formulations.

All the more surprising is the experimental finding that the compositions according to the invention avoid side effects that exists with other statin sustained release formulations.

One example of the surprising experimental findings is that the slower release of HMG-CoA reductase inhibitor (e.g. pitavastatin) from matrix tablet obtained by increasing hydrophilic polymer viscosity (HPMC) permits the decrease of pitavastatin plasma level and surprisingly avoids undesirable muscular side effect while still delivering at target organ (the liver) the drug concentration leading to optimal drug efficacy.

The composition according to the invention show further surprising beneficial effects e.g., an improved efficacy with low does of active agent compared to other statin sustained release formulation.

The term ‘modified’, “extended” “sustained release” hereinbefore and hereafter shall corresponds to an active ingredient that is released from the dosage form over an extended period of time, for example greater than about four hours. Preferably, the pharmaceutical compositions release less than about 80 weight percent of the active agent in the first eight hours after ingestion of the composition, with the balance of the pharmaceutically active agent being released thereafter. In preferred compositions, less than about 15 weight percent of the pharmaceutically active agent is released in the first 0.5 hour after ingestion, from about 10 to about 50 weight percent of the pharmaceutically active agent is released within about 2 hours after ingestion, and from about 40 to about 90 preferably about 40 to about 80 weight percent of the pharmaceutically active agent is released within about 6 hours after ingestion.

HMG-CoA reductase inhibitors, also called □-hydroxy-□methylglutaryl-co-enzyme-A reductase inhibitors (and also called statins) are understood to be those active agents which may be preferably used to lower the lipid levels including cholesterol in blood and can be used e.g. for the prevention or treatment of hyperlipidemia and artheriosclerosis.

The class of HMG-Co-A reductase inhibitors comprises compounds having differing structural features.

Preferred are compounds which are selected from the group consisting of atorvastatin, cerivastatin, fluvastatin, lovastatin, pitavastatin (formerly itavastatin), rosuvastatin, and simvastatin, or, in each case, a pharmaceutically acceptable salt thereof.

Especially preferred HMG-Co-A reductase inhibitors are those agents which have been marketed. Most preferred are atorvastatin, fluvastatin, pitavastatin, rosuvastatin or simvastatin or a pharmaceutically acceptable salt thereof, in the first line pitavastatin or a pharmaceutically acceptable salt thereof.

Only salts that are pharmaceutically acceptable and non-toxic are used therapeutically and those salts are therefore preferred.

The corresponding active ingredient or a pharmaceutically acceptable salt thereof may also be used in form of a solvate, such as a hydrate or including other solvents, used for crystallization.

The structure of the active agents identified hereinbefore or hereinafter by generic or trade names may be taken from the actual edition of the standard compendium “The Merck Index” or from databases, e.g. IMS Life cycle (e.g. IMS World Publications). The corresponding content thereof is hereby incorporated by reference. Any person skilled in the art is fully enabled to identify the active agent and, based on these references, likewise enabled to manufacture and test the pharmaceutical indications and properties in standard test models, both in vitro and in vivo.

In a preferred embodiment of the present invention the amount of an HMG-CoA reductase inhibitor or pharmaceutically acceptable salt thereof is about 5 to 50% by weight based on the total core components, preferably about 5 to 21, e.g. about 5% wt, about 10% wt, about 11%, about 20% wt, about 21% wt by weight based on the total core components.

In another preferred embodiment of the present invention the amount of an HMG-CoA reductase inhibitor or pharmaceutically acceptable salt thereof is about 1 to 50% by weight based on the total core components, preferably about 1 to 21, e.g. about 1% wt, about 3% wt, about 4% wt, about 5% wt, about 6% wt, about 7% wt, about 8% wt, about 10% wt, about 11%, about 15%, about 16%, about 17%, about 20% wt, about 21% wt by weight based on the total core components.

In an especially preferred embodiment of the invention the amount of an HMG-CoA reductase inhibitor (especially pitavastatin calcium) or pharmaceutically acceptable salt thereof is about 1-32 mg, preferably 1-17 mg per dosage unit form.

In an embodiment of the invention the inner phase of the composition can comprise a matrix former.

Hydrophilic and/or hydrophobic components can be used as matrix former.

Hydrophilic, non-ionic, slowly swelling and gel forming polymers are employed as matrix former. These polymers exhibit different swelling characteristics and therefore different viscosities in aqueous media and form upon ingestion of the solid dosage form different diffusion barriers (the matrix) releasing the drug substance by rate-controlled diffusion of the drug substance through these diffusion barriers. A substantial amount of the released active agent may be processed efficiently at the targeted active site. The non-ionic, hydrophilic polymer is present in an amount providing sufficient strength to the gel matrix to prevent its premature degradation. The gel matrix should also be formed within a time period that is effective to prevent the premature release of the active agent.

For example, the gel matrix preferably forms within about 5 minutes after ingestion of the composition to prevent a burst of active agent prior to gel formation. It has turned out that the nonionic, hydrophilic polymer operates to decrease the rate of gel formation to an acceptable level. The non-ionic, hydrophilic polymer may be present in the pharmaceutical composition in an amount ranging from about 1 to about 80 weight percent, preferably from about 1 to about 60 weight percent, more preferably from about 15 to about 50% by weight based on the total core components, most preferably from about 18 to about 40% by weight based on the total core components.

The matrix former can be selected from the group consisting of, polyethylene glycol, polyvinylpyrrolidone, polyvinyl alcohol, and hydrophilic polymers such as hydroxypropyl methyl cellulose (HPMC), hydroxypropylcellulose and hydroxymethylcellulose.

The matrix former can furthermore be selected from the group consisting of polysaccharides such as alginate, carrageenan, scleroglucan, pullulan, dextran, hyaluronic acid, chitin, chitosan and starch.

The matrix former can furthermore be selected from the group consisting of natural polymers such as proteins, for example, albumin or gelatine, and natural rubber.

The matrix former can furthermore be selected from the group consisting of synthetic polymers such as acrylates, for example, polymethacrylate, poly(hydroxy ethyl methacrylate), poly(methyl methacrylate), poly(hydroxy ethyl methacrylate-co-methyl metacrylate, Carbopol 934™, polamides such as polyacrylamide or poly(methylene bis acrylamide), polyanhydrides such as poly(biscarboxyphenoxy)methane, PEO-PPO block-co-polymers such as poloxamers, polyvinyichloride, polyvinyl pyrrolidone, polyvinyl alcohol, polyethylene, polyethylene glycols and co-polymers thereof, polyethylene oxides and co-polymers thereof, polypropylene and co-polymers thereof, polystyrene, polyesters such as poly(lactic acid), poly(glycolic acid), poly(caprolactone) and co-polymers thereof, poly(ortho esters and co-polymers thereof, resins such as Dowex™ or Amberlite™, polycarbonate, cellophane, silicones such as poly(dimethylsiloxane), polyurethanes, and synthetic rubbers such as styrene butadiene rubber or isopropene rubber.

The matrix former can furthermore be selected from the group consisting of shellacs, waxes such as carnauba wax, beeswax, glycowax or castor wax, nylon, stearates such as glycerol palmitostearate, glyceroyl monostearate, glyceryl tristearate or stearyl alcohol, lipids such as glycerides or phospholipids, and paraffin.

In a most preferred embodiment of the present invention an HPMC is selected as matrix former.

In a preferred embodiment of the present invention the pharmaceutical compositions comprise from about 1 to about 60% of HPMC by weight based on the total core components, preferably from about 15 to 50% of HPMC by weight based on the total core components, more preferably from about 18 to about 40% of HPMC by weight based on the total core components.

The HPMC components have an average molecular weight ranging from approximately 20,000 to approximately 170,000. These molecular weights correspond to viscosities of approximately 1 to approximately 100,000 cps (viscosities values given of 2% aqueous solutions of the HPMC types).

According to the invention, the matrix former may comprise one or more type(s) of matrix former(s) having different viscosities.

In a preferred embodiment of the present invention the matrix former has a viscosity of about 1 to about 100 000 cps, e.g from about 1 to 4000 cps, preferably of about 1 to about 500 cps, preferably of about 1 to about 250 cps, more preferably of about 1 to about 125 cps.

In a preferred embodiment, the total amount of the matrix forming HPMC component(s) present in the internal phase, having a viscosity of about 100 cps ranges from about 0-60 mg, preferably from about 10-60 mg, more preferably 10-35 mg per dosage unit form.

In another especially preferred embodiment of the invention, the matrix forming HPMC components are selected from the group consisting of HPMC K100LVP CR 100 cps used in the internal phase (also named Methocel K100 Premium LVCR EP (100 cps)).

In another preferred embodiment, the total amount of the matrix forming HPMC component(s) present in the internal phase, having a viscosity of about 100 000 cps ranges from about 10-60 mg, preferably from about 10-40 mg, more preferably 10-35 mg per dosage unit form.

In another especially embodiment of the invention, the matrix forming HPMC components of the internal phase are a combination of a matrix forming HPMC having a viscosity of about 100 000 cps and a matrix forming HPMC having a viscosity of about 100 cps.

The composition according to the present invention furthermore may also comprise a stabilizer, especially for protecting the drug substance adequately against pH-related destabilization.

Additionally, the heat and light sensitivity as well as hygroscopicity of an active ingredient impose particular requirements on the manufacture and storage of pharmaceutical dosage forms.

Certain HMG-CoA reductase inhibitors are extremely susceptible to degradation at pH below about 8. An example of such a compound comprises the compound having the USAN designation fluvastatin sodium (hereinafter “fluvastatin”), of the chemical designation: R*,S*-(E)(±)-7-[3-(4-fluorophenyl)-1-(1-methyl-ethyl)-1H-indol-2-yl]-3,5-dihydroxy-6-heptenoic acid, sodium salt, [see European Patent Application EP-A-114027].

For example, the degradation kinetics of fluvastatin in aqueous solution at various pH is illustrated below:

% fluvastatin remaining at 37° C.

| pH | after 1 hour | after 24 hrs |
|---|---|---|
| 7.8 | 98.3 | 98.0 |
| 6.0 | 99.6 | 97.1 |
| 4.0 | 86.7 | 25.2 |
| 1.0 | 10.9 | 0 |

The above-indicated instability of fluvastatin and related HMG-CoA reductase compounds is believed to be due to the extreme lability of the β,δ-hydroxy groups on the heptenoic acid chain and the presence of the double bond, such that at neutral to acidic pH, the compounds readily undergo elimination or isomerization or oxidation reactions to form conjugated unsaturated aromatic compounds, as well as the threo isomer, the corresponding lactones, and other degradation products.

In order to achieve marketable dosage forms that meet the international quality criteria (e.g. for approval) comprising HMG-CoA reductase inhibitor compound, it is essential to adequately protect it against pH-related destabilization by using a stabilizer.

A preferred stabilizer to be used according to the present invention is an “alkaline medium”, said alkaline medium being capable of stabilizing the composition by imparting a pH of at least 8 to an aqueous solution or dispersion of the composition. Since the stabilizer is added in solution during the aqueous granulation process, it is in intimate contact with the active ingredient in the composition to achieve optimal stability of the medicament.

The term “alkaline medium” or “base” employed herein shall refer to one or more pharmaceutically acceptable substances capable of imparting a pH of at least 8, and preferably at least 9, and up to about pH 10, to an aqueous solution or dispersion of the composition of the invention. More particularly, the alkaline medium creates a “micro-pH” of at least 8 around the particles of the composition when water is adsorbed thereon or when water is added in small amounts to the composition. The alkaline medium should otherwise be inert to the composition compounds. The pH may be determined by taking a unit dosage of the composition containing e.g. 4 mg of pitavastatin or the equivalent amount of another compound and dispersing or dissolving the composition in 10 to 100 ml of water. The pharmaceutically acceptable alkaline substance(s) which comprise the alkaline medium may range from water-soluble to sparingly soluble to essentially water-insoluble.

In a preferred embodiment of the present invention, the stabilizer is a basic stabilizer selected from the group consisting of inorganic water-soluble or inorganic water-insoluble compound.

An inorganic water-soluble compound is a suitable carbonate salt such as sodium or potassium carbonate, sodium bicarbonate, potassium hydrogen carbonate, phosphate salts selected from, e.g., anhydrous sodium, potassium or calcium dibasic phosphate, trisodium phosphate, alkali metal hydroxides, selected from sodium, potassium, or lithium hydroxide, and mixtures thereof.

Sodium bicarbonate advantageously serves to neutralize acidic groups in the composition in the presence of moisture that may adsorb onto particles of the composition during storage. The calcium carbonate exerts a buffering action in the stored composition, without apparent effect on drug release upon ingestion. It has further been found that the carbonate salts sufficiently stabilize the drug substance such that conventional water-based preparative techniques, e.g. trituration with water or wet granulation, can be utilized to prepare stabilized compositions of the invention.

Examples of water-insoluble compound are suitable alkaline compounds capable of imparting the requisite basicity include certain pharmaceutically acceptable inorganic compounds commonly employed in antacid compositions (e.g., magnesium oxide, hydroxide or carbonate; magnesium hydrogen carbonate; aluminum or calcium hydroxide or carbonate; composite aluminum-magnesium compounds, such as magnesium aluminum hydroxide); as well as pharmaceutically acceptable salts of phosphoric acid such as tribasic calcium phosphate; and mixtures thereof.

In a preferred embodiment of the invention, the stabilizer is an inorganic water insoluble suitable silicate compound such as magnesium aluminometasilicate (neusilin). Said stabilizer can be introduced in the manufacturing process in the internal phase or in the external phase. Studies showed that neusilin has a higher stabilizing effect than some inorganic water-soluble stabilizers.

The proportion of a particularly stabilizing excipient to be employed will depend to some extent on the intended manufacturing process. In compositions to be tableted, for example, calcium carbonate should not exceed a proportion which can no longer be conveniently subjected to compression, and will generally be used in combination with a more readily compressible alkaline substance, e.g., sodium bicarbonate. On the other hand, capsule dosage forms may comprise higher levels of poorly compressible excipients, provided that the overall composition remains sufficiently free-flowing and processable.

In a preferred embodiment, the amount of the stabilizer is about 1-15 weight % of the composition.

In a preferred embodiment, the amount of stabilizer is from about 0.1-10 mg per dosage unit. An example of a stabilized composition according to the invention may comprise in weight percent based on the total core components: 0.1 to 60 weight % (wt. %), typically 0.5 to 40 wt. %, of the active ingredient (e.g., pitavastatin); and preferably 0.1 to 35 wt. %, more preferably 1-15 wt. % (e.g. 1 wt %, 1.25 wt %, 2 wt %, 3 wt %, 4 wt %), of water insoluble compound such as neusilin or soluble carbonate compound, for example, selected from potassium bicarbonate, potassium carbonate and/or mixtures thereof.

In another embodiment the stabilized composition according to the invention may comprise in weight percent based on the total core components most preferably about 1-21 wt. % of the active ingredient (e.g., pitavastatin) e.g about 1 wt %, 1.25 wt %, about 2 wt %, about 3 wt %, about 4 wt %, about 5 wt %, about 6 wt %, about 7 wt %, 8 about wt %, about 9 wt %, about 10 wt %, about 11 wt %, about 12 wt %, about 13 wt %, about 14 wt %, about 15 wt %, about 16 wt %, about 17 wt %, about 18 wt %, about 19 wt %, about 20 wt %, about 21 wt %; and preferably 0.1 to 35 wt. %, more preferably 1-15 wt. % (e.g. 1 wt %, 1.25 wt %, 2 wt %, 3 wt %, 4 wt %), of water insoluble compound such as neusilin or soluble carbonate compound, for example, selected from potassium bicarbonate, potassium carbonate and/or mixtures thereof.

It is a further advantage that the stabilized compositions of the invention can be readily prepared by aqueous or other solvent-based techniques, e.g. wet granulation.

The resulting composition has been found to provide an extended storage life of the HMG-CoA reductase inhibitor compounds, even in the presence of moisture or when such compositions additionally comprise otherwise potentially reactive excipients, such as lactose.

The drug substance in compositions of the invention was proven to be at least stable during 18 months at 25° C. (assays between 98% and 99%, after 18 months at 25° C.).

Compositions also having particularly attractive storage stability comprise, as an alkaline medium, both a water-soluble alkaline excipient and a water-insoluble or sparingly soluble alkaline excipient.

A solid unit dosage composition may have the ratio of water insoluble to soluble carbonate from e.g. 40:1 to 1:2.

An exemplary tablet of the invention may comprise a ratio between calcium carbonate and sodium bicarbonate of about 2:1 to 1:2 by weight. A capsule composition may comprise these excipients in a ratio of, for example, 25:1 to 35:1 by weight.

The composition according to the present invention may furthermore comprise a filler. In addition to the drug substance and alkaline medium, a filler is also generally employed in the compositions to impart processability. Suitable filler materials are well-known to the art (see, e.g., Remington's Pharmaceutical Sciences, 18th Ed. (1990), Mack Publishing Co., Easton, Pa., pp. 1635-1636), and include microcrystalline cellulose, lactose and other carbohydrates, starch, pregelatinized starch, e.g., starch 1500 (Colorcon Corp.), corn starch, dicalcium phosphate, potassium bicarbonate, sodium bicarbonate, cellulose, calcium phosphate dibasic anhydrous, sugars, sodium chloride, and mixtures thereof, of which lactose, microcrystalline cellulose, pregelatinized starch, and mixtures thereof, are preferred.

Owing to its superior disintegration and compression properties, microcrystalline cellulose (Avicel PH1, Avicel R, FMC Corp.), and mixtures comprising microcrystalline cellulose and one or more additional fillers, e.g., pregelatinized starch, are particularly useful.

The total filler is present in the compositions in an amount of about 1 to 65 wt. % by weight based on the total core components, preferably 20 to 60 wt %, more preferably 30 wt % to 50 wt % by weight based on the total core components.

The invention relates to compositions wherein the total amount of the filler is from about 20-60 mg, preferably from about 20 to 45, more preferably 25-45 mg per dosage unit and preferably consists of microcrystalline cellulose.

The composition according to the present invention comprises film coating components. The drug release profiles from matrix tablets are very much determined by the accelerated drug release in acidic media, e.g. in the gastric juice. Therefore there was a need to formulate enteric coated variant.

The coating according to the invention is not used to avoid acidic pH instability because the compositions according to the inventions are relatively stable in acidic pH, but to avoid the drug release in acidic media and trigger the drug release in the small intestine where the pH is higher and where the drug release is slower.

Enteric film coating components are applied to oral tablets, pellets or capsules to protect against a premature fast release of the drug substance in the stomach prior to reaching the intestinal absorption site where the drug release is slower.

As illustration, Table 1 discloses the release profiles of two non-coated pitavastatin extended release formulations X205 and X203 which have been measured at pH 1 (stomach conditions) and at pH 6.8 (intestine conditions).

pH 1
Batch X205

| | Time (min) | | | | | | | |
|---|---|---|---|---|---|---|---|---|
| | 30 | 60 | 120 | 240 | 360 | 480 | 600 | 720 |
| MEAN (% of active | 24 | 37 | 57 | 85 | 96 | 99 | 99 | 99 |
| released) | | | | | | | | |

pH 6.8
Batch X205

| | Time (min) | | | | | | |
|---|---|---|---|---|---|---|---|
| | 30 | 60 | 120 | 240 | 360 | 480 | 600 |
| MEAN (% of active | 14 | 26 | 45 | 74 | 93 | 102 | 103 |
| released) | | | | | | | |

pH 1
Batch X203

| | Time (min) | | | | | | | | | |
|---|---|---|---|---|---|---|---|---|---|---|
| | 30 | 60 | 120 | 240 | 360 | 480 | 600 | 720 | 840 | 960 |
| MEAN (% of active | 17 | 29 | 47 | 70 | 84 | 93 | 97 | 99 | 100 | 100 |
| released) | | | | | | | | | | |

pH 6.8
Batch X203

| | Time (min) | | | | | | | | | |
|---|---|---|---|---|---|---|---|---|---|---|
| | 30 | 60 | 120 | 240 | 360 | 480 | 600 | 720 | 840 | 960 |
| MEAN (% of active | 8 | 13 | 24 | 42 | 58 | 71 | 83 | 93 | 100 | 104 |
| released) | | | | | | | | | | |

Table 1 shows that for both non-coated pitavastatin extended release formulations X203 and X205, the drug release is faster at pH 1 than at pH 6.8 as can be inferred from the higher solubility of pitavastatin in acidic pH conditions.

Table 2 discloses data obtained by measuring drug dissolution (% wt) from an enteric coated tablet which first stayed 2 hours (120 minutes) at pH1 (HCl 0.1 N) and which was then transferred at pH 6.8 (phosphate buffer) and stayed at pH 6.8 from 120 minutes to 720 minutes (The pH is shifted from 1 to 6.8 after 120 minutes).

After 120 minutes at pH1 no drug has been released (see table below). The release starts when the tablet is transferred to pH 6.8 (at 120 minutes)

Batch X180

| | Time (min) | | | | | | | |
|---|---|---|---|---|---|---|---|---|
| | 120 | 150 | 180 | 240 | 360 | 480 | 600 | 720 |
| MEAN (% of | 0 | 6 | 15 | 29 | 56 | 76 | 85 | 91 |
| active released) | | | | | | | | |

The data should be read as follows: 0% of the drug was released after 120 minutes, 6% was released after 150 minutes, 15% was released after 180 minutes.

These data show an absence of release of HMG-CoA reductase inhibitor from the enteric coated composition at acidic pH (at least during 2 hours). Since the active release occurs at pH 6.8 for an enteric coated variant, it is much slower than the release of a non-coated variant which occurs in the stomach under acidic conditions.

In vivo the enteric coated variant starts to release the drug in the intestine, i.e. at pH around 5.5-6 while the non-functionally coated variant releases the drug in the stomach, i.e. at acidic pH around pH1-3.

The In Vivo release profiles of the non-functionally coated pitavastatin extended release formulations variant (variant which is coated with a film dissolving in water whatever the pH conditions) vary with the residence time of the extended release formulation in the stomach where the tablets are submitted to mechanical forces. In Vivo studies have shown that this is leading to an unfavorable food effect.

This disadvantage does not exist with pitavastatin (NKS104) enteric-coated sustained release formulation whose release profile is not affected by the gastric juice, less affected or not affected by the residence time in the stomach, and less affected or not affected by the mechanical forces in the stomach. In Vivo studies have shown that for the enteric coated formulation, the drug plasma levels (AUC and Cmax) in the fed state are not increased as compared to the fasted state.

Furthermore, the combination of a controlled release matrix tablet with an enteric coat may have led to an incomplete release of the drug substance in the intestinal tract and a loss of absorption if the release time had exceeded the intestinal transit time. However no decrease of bioavailability was observed in the fasted state as compared to the non-functionally coated formulation.

In a preferred embodiment the core of the composition is first coated with a non functional film coat and then with an enteric coat.

The enteric coat is a film insoluble at acidic pH and which dissolves when pH increases above pH 5-5.5, i.e. as soon as the formulation passes the pylorus.

In a preferred embodiment the film coat contains Methacrylic acid copolymers (type C USP) as enteric film former, Polyethylene glycol and Triethylcitrate as plasticizers, Sodium carboxymethylcellulose as suspending agent, Talc and Pigments.

Examples of enteric film coat include hydroxypropylmethylcellulose phthalate, cellulose acetate phthalate, polyvinyl acetate phthalate, methylcellulose phthalate, copolymerized methacrylic acid/methacrylic acid methyl esters (e.g., Eudragit®, Rohm Pharma). The enteric coating is preferably applied to result in about a 5 to 12, preferably 7 to 10, weight percent increase of the capsule, pellet or tablet core. (this is usually expressed as 4 to 6 mg/cm2)

Tableted compositions of the invention are desirably coated to protect against moisture and light discoloration, and to mask the bitter taste of the drug. Either the enteric coating may contain opacifiers and colorants, or a conventional opaque film coating may be applied to the tablet core, optionally after it has been coated with an enteric substance.

Other conventional enteric or film coating composition ingredients include plasticizers, e.g., polyethylene glycol (e.g. polyethylene glycol 6000), triethylcitrate, diethyl phthalate, propylene glycol, glycerin, butyl phthalate, in conventional amounts, as well as the above-mentioned opacifiers such as titanium dioxide, and colorants, e.g. iron oxide, aluminum lakes, etc.

Non functional film coat to be applied to compositions of the invention comprise, e.g., polyethylene glycol, polyvinylpyrrolidone, polyvinyl alcohol, hydrophilic polymers such as hydroxypropylcellulose, hydroxymethylcellulose, and hydroxypropylmethylcellulose or the like, of which hydroxypropylmethylcellulose (e.g., Opadry, Colorcon Corp.) is preferred. Hydrophobic film-formers which may be applied using an organic solvent vehicle comprise, for example, ethyl cellulose, cellulose acetate, polyvinyl alcohol-maleic anhydride copolymers, etc.

The film coating may be generally applied to achieve a weight increase of the pellet or core or tablet of about 1 to 10 wt. %, and preferably about 2 to 6 wt. %.

The enteric or film coatings can be applied by conventional techniques in a suitable coating pan or fluidized bed apparatus using water and/or conventional organic solvents (e.g., methyl alcohol, ethyl alcohol, isopropyl alcohol), ketones (acetone, ethylmethyl ketone), chlorinated hydrocarbons (methylene chloride, dichloroethane), etc.

It has been surprisingly found that the non functional sublayer placed between the core component and the enteric coat protects the active from chemical degradation caused by direct contact with the enteric coat.

This property is exemplified in the table 3 below.

Batch JO-4286.05A is a sustained release pitavastatin formulation coated only with enteric coat.

Batch JO-4286.06A is a sustained release pitavastatin formulation coated with subcoat (non functional coat) and an enteric coat.

The stability study shows that the degradation product lactone is forming faster without the protective subcoat.

TABLE 3

| Batch | Storage | | Assay [%] | Degradation products [%] | | | |
|---|---|---|---|---|---|---|---|
| Neusilin | conditions | Time | NKS104 | Lactone | Ketone | unknown | sum |
| JO-4286.05A | 5° C. | 6 w | 98.5 | <0.1 | 0.2 | <0.1 | 0.2 |
| 3.2 mg | 40° C./75% | 6 w | 99.0 | 0.1 | 0.2 | <0.1 | 0.3 |
| | 40° C./75% open | 6 w | 98.3 | 0.2 | 0.2 | <0.1 | 0.4 |
| | | 3 m | 97.1 | 0.3 | 0.2 | <0.1 | 0.5 |
| | | 6 m | 96.6 | 0.4 | 0.2 | <0.1 | 0.5 |
| | 50° C. | 6 w | 97.8 | 0.2 | 0.2 | <0.1 | 0.4 |
| | | 3 m | 96.6 | 0.2 | 0.2 | <0.1 | 0.4 |
| | | 6 m | 96.3 | 0.3 | 0.2 | <0.1 | 0.5 |
| JO-4286.06A | 5° C. | 6 w | 99.1 | <0.1 | 0.2 | <0.1 | 0.2 |
| 3.2 mg + | 40° C./75% | 3 m | 98.8 | <0.1 | 0.2 | <0.1 | 0.3 |
| additional | | 6 m | 97.0 | 0.2 | 0.2 | <0.1 | 0.4 |
| HPMC coat | 40° C./75% open | 6 w | 94.7 | 0.1 | 0.2 | <0.1 | 0.3 |
| (bi-layer) | | 3 m | 97.1 | 0.1 | 0.2 | <0.1 | 0.3 |
| | | 6 m | 97.6 | 0.2 | 0.2 | <0.1 | 0.4 |
| | 50° C. | 6 w | 98.3 | 0.1 | 0.2 | <0.1 | 0.3 |
| | | 3 m | 98.0 | 0.2 | 0.2 | <0.1 | 0.4 |
| | | 6 m | 97.9 | 0.3 | 0.2 | <0.1 | 0.5 |

This reveals that the sustained release pitavastatin formulation coated with subcoat (non functional coat) and an enteric coat is more stable than the sustained release pitavastatin formulation coated only with enteric coat.

The composition according to the present invention may furthermore comprise further components.

Further components which may be incorporated into the compositions to facilitate processing and/or provide enhanced properties of the product dosage form, are selected from the group consisting of:

a) well-known tabletting binders (e.g., hydroxypropylmethylcellulose, starch, starch pregelatinized (starch 1500), gelatin, sugars, natural and synthetic gums, such as carboxymethylcellulose, methylcellulose, polyvinylpyrrolidone, low substituted hydroxypropylcellulose, ethylcellulose, polyvinylacetate, polyacrylates, gelatin, natural and synthetic gums), microcrystalline cellulose, and mixtures of the foregoing;

b) disintegrants (e.g., cross-linked carboxymethyl-cellulose, croscarmelose, crospovidone, sodium starch glycolate);

c) lubricants (e.g., magnesium stearate, stearic acid, calcium stearate, glyceryl behenate, hydrogenated vegetable oil, carnauba wax and the like);

d) flow agents (e.g., silicon dioxide, talc, polyethylene oxides);

e) anti-adherents or glidants (e.g., talc)

f) sweeteners;

g) coloring mediums (e.g., iron oxide, aluminum lakes);

h) flavoring mediums;

i) antioxidants, etc.

Selection of a particular ingredient or ingredients and the amounts used will be readily determinable by one skilled in the art by reference to standard procedures and practices for preparing tablets or capsules or other dosage forms.

In general, an effective amount of a tabletting binder will comprise about 1 to 10 wt. %, and preferably 1 to 5 wt. %; anti-adherents or glidants, about 1 to 10 wt. %; disintegrants, about 1 to 5 wt. %, and lubricants, about 0.1 to 2 wt. %, by weight based on the total core components.

A composition according to the invention comprises (in weight percent based on the total core components):

a) Drug substance: approx. 5-50 wt % of the formulation; preferably 5-20 wt %, for example 10-20 wt %, e.g. about 10 wt %, e.g. about 11 wt %

b) Matrix former: The amount of HPMC as matrix former is between 1 to 80 wt %, preferably between 15 and 70 wt %, more preferably 20-70 wt %

c) Stabilizer (alkaline medium): 1-15 wt %

d) Filler: About 1 to 65 wt %, preferably about 20-60 wt %, more preferably approx. 50 wt %.

e) coat:

non functional coat: used at about 4 mg of film coat pro cm2,
enteric coating: used at 4 to 6 mg polymer pro cm2

In another embodiment, a composition according to the invention comprises (in weight percent based on the total core components):

a) Drug substance: approx. 5-50 wt % of the formulation; preferably about 1-21 wt. % of the active ingredient (e.g., pitavastatin) e.g about 1 wt %, 1.25 wt %, about 2 wt %, about 3 wt %, about 4 wt %, about 5 wt %, about 6 wt %, about 7 wt %, 8 about wt %, about 9 wt %, about 10 wt %, about 11 wt %, about 12 wt %, about 13 wt %, about 14 wt %, about 15 wt %, about 16 wt %, about 17 wt %, about 18 wt %, about 19 wt %, about 20 wt %, about 21 wt %;

b) Matrix former The amount of HPMC as matrix former is between 1 to 80 wt %, preferably between 15 and 70 wt %, more preferably 20-70 wt %

c) Stabilizer (alkaline medium): 1-15 wt %

d) Filler: About 1 to 65 wt %, preferably about 20-60 wt %, more preferably approx. 50 wt %.

e) coat:

non functional coat: used at about 4 mg of film coat pro cm2,
enteric coating: used at 4 to 6 mg polymer pro cm2

The inner phase of the pharmaceutical composition according to the invention can comprise the drug substance, a filler, a binder a stabilizer, and a matrix former.

The outer phase of the pharmaceutical composition according to the invention can comprise a flow agent, a lubricant, and optionally a filler.

The core components are first coated with a non functional film coat and then with an enteric coat.

In a preferred embodiment the drug substance consists in pitavastatin Ca-salt.

The drug substance is used preferably from 5 wt % to 20 wt % by weight based on the total core components, e.g about 5.225 w %, e.g at about 10%, e.g. 10.45 wt %, e.g about 21 wt %.

In a preferred embodiment the filler consists in microcrystalline cellulose.

The total amount of filler is used preferably at about 50% by weight based on the total core components.

In a most preferred embodiment, the filler of the internal phase is used at about 20-52 wt % by weight based on the total core components e.g. at about 26.05 wt %, about 38 wt %, about 39 wt %, about 39.8 wt %, about 43 wt %, about 44.8 wt %, about 46, 67 wt %, about 48 wt %, about 51.05 wt %, about 53 wt % by weight based on the total core components.

In a most preferred embodiment, the filler of the external phase is used at about 15-20 wt % by weight based on the total core components, e.g. 18.75 wt % by weight based on the total core components.

In a preferred embodiment the binder consists in low substituted hydroxypropylcellulose HPC or hydroxypropylmethylcellulose HPMC (e.g. 3 or 6 cps).

In a preferred embodiment, the binder is used at about 1-10 wt % by weight of the core components, e.g. 10 wt %, most preferably 1-5 wt %, e.g. at about 3, 125 wt %, or 5 wt % by weight based on the total core components.

In a preferred embodiment the stabilizer consists in potassium bicarbonate or magnesium aluminometasilicate (neusilin).

In a preferred embodiment the stabilizer is used at about 1-15 wt % by weight based on the total core components e.g. at about 1.25 wt %. e.g. at about 4% by weight based on the total core components.

In a preferred embodiment the matrix former of the internal phase consist in HPMC having a viscosity of about 100 cps and used at about 15-50 wt % by weight based on the total core components.

In a most preferred embodiment the matrix former of the internal phase is used at a bout 30 wt %, e.g 31.25 wt % by weight based on the total core components.

In a preferred embodiment the flow agent consists in silicon dioxide colloidal (e.g. Aerosil).

In a preferred embodiment the flow agent is used at about 0.1-2 wt %, e.g. 0.5 wt % by weight of the core components.

In a preferred embodiment the lubricant consists in magnesium stearate.

In a preferred embodiment the lubricant is used at about 0.1-2 wt %, e.g. 0.5 wt % by weight of the core components.

In a preferred embodiment, the first layer of the bilayer coating consists in a non-functional coat consisting in Hydroxypropylmethylcelluloce (film former), Polyethyleneglycol (plasticizer), pigment (for example titanium dioxide) and a lubricant (talc).

In a preferred embodiment non-functional coat is used at about 4 mg of film coat pro cm2,

In a preferred embodiment, the enteric coat of the bilayer coating consists in Eudragit L30D (methacrylic copolymer), talc and polyethyleneglycol.

In a preferred embodiment enteric coat is used at 4 to 6 mg polymer pro cm2.

The invention particularly relates to compositions wherein the ratio between the matrix forming HPMC and the total weight, is from about 0.20:1 to about 0.35:1, preferably from 0.25:1 to about 0.35:1 e.g 0, 31:1, 0.30:1, 0.27:1.

The present invention is concerned with compositions wherein the ratio between the matrix forming HPMC total and the HMG-CoA reductase inhibitor is from about 1:1 to about 10:1 preferably about 1:1 to about 6:1 e.g 5, 99:1, 3:1, 1, 5:1.

Furthermore the invention relates to a composition wherein the ratio between the filler in the internal phase and the matrix former HPMC comprised in the internal phase is from about 1:1 to about 2:1 e.g 1.2:1, 1, 7:1, 1, 5:1

The present invention is concerned with compositions wherein the ratio between the stabilizer and the total core weight (without coating) is from about 0.001:1 to about 0.01:leg. 0.004:1, 0.003:1.

The present invention is concerned with compositions wherein the ratio between the stabilizer and the HMG-CoA reductase inhibitor is from about 0.1:1 to about 1:, e.g 0.2:1, 0.4:1, 0.8:1.

The present invention is concerned with compositions wherein the ratio between the flow agent and the total core weight (without coating) is from about 0.001:1 to about 0.01:1, e.g 0.004:1, 0.005:1.

The present invention is concerned with compositions wherein the ratio between the lubricant and the total core weight (without coating), is from about 0.001:1 to about 0.01:1 e.g 0.004:1, 0.005:1.

The present invention is concerned with compositions wherein the ratio between the non functional coat and the total core weight (without coating) is from about 0.01:1 to about 0.1:1 e.g 0.04:1, 0.05:1

The present invention is concerned with compositions wherein the ratio between the enteric coat and the total core weight (without coating),) is from about 0.01:1 to about 0.1:1 e.g 0.06:1, 0.07:1, 0.075:1

To obtain very stable compositions, an aqueous or other solvent-based preparative process is preferably utilized, whereby the drug substance and alkaline medium are blended together in the presence of minor amounts of, e.g., water, to provide particles containing the drug and alkaline substance in intimate admixture. The solvent or liquid dispersion medium can be for example, water, ethanol, a polyol (for example, glycerol, propylene glycol, liquid polyethylene glycols, and the like), vegetable oils, nontoxic glyceryl esters, and suitable mixtures thereof. Given the hygroscopicity and moisture sensitivity of HMG-CoA reductase inhibitor compounds such as pitavastatin, it is unexpected that the drug substance is sufficiently stabilized by the alkaline medium to resist degradation by a such techniques.

In another embodiment of a solvent-based process which can assist subsequent drying in a fluidized bed, the drug substance and alkaline medium are wet granulated by known techniques, i.e. blended in the moistened state, together with an amount of the filler material. The thus-formed granules, after drying, are then combined with any remaining filler and other set-asides, e.g., binder, lubricant, and can therefore be tableted, encapsulated, or otherwise shaped into a dosage form.

Drying is conventionally performed by tray drying or in a fluidized bed, preferably the latter.

It has been found that a water-soluble stabilizing alkaline substance such as sodium carbonate or bicarbonate or other alkaline medium, can be added in situ to the above-described aqueous phase comprising the pitavastatin or other HMG-CoA reductase inhibitor compound, and upon subjecting this aqueous phase to a freeze-drying procedure, there can be obtained particles comprising the drug compound co-lyophilized with the added alkaline substance.

Very good contacting of the drug and stabilizer can thereby be achieved, to the extent that stable compositions of the invention may be prepared, for example, from the drug and sodium carbonate in a weight ratio of about 10/1 to 100/1. For example, a co-lyophilized composition of the invention comprising as low as 0.1% by weight sodium carbonate has been found effective to provide a highly stabilized drug composition.

Enteric or film coating of a microcrystalline cellulose-based tablet with a water-based film coating composition is desirably carried out at a bed temperature of 30-50° C., an inlet temperature of 50-80° C. and a relative humidity (RH) of less than 50%.

The resulting tableted or capsule dosage forms should be protected during storage against thermal or light induced oxidation as well as moisture contamination.

Pharmaceutical compositions, e.g. oral dosage forms, according to the invention may be formulated in any conventional form, e.g. powders, granules/granulates, capsules or tablets. Preferred pharmaceutical compositions may be in the form of tablets.

The pharmaceutical composition according to the invention may have a dosage weight from about 5 to about 300 mg, preferably about 100 mg, more preferably about 80 mg.

Such compositions may be formulated by known means to provide standard unitary oral dosages of compound, e.g. 3 mg, 4 mg, 6 mg, 8 mg, 12 mg, 16 mg, etc., as e.g., powders, granulates, capsules, pellets or tablets.

A special embodiment of the invention relates to tablet having a diameter from 4 to 8 mm, for example from 6 to 8 mm having a weight between 70 to 180 mg wherein the active ingredient has a weight between 4 and 40 mg per dosage unit form

Another special embodiment of the invention relates to tablet having a diameter from 4 to 8 mm, for example from 6 to 8 mm having a weight between 70 to 180 mg wherein the active ingredient has a weight between 3 and 40 mg per dosage unit form

Pharmaceutical compositions, e.g. oral dosage forms, hereinabove described may be formed of a granulated mass comprising, pitavastatin HPMC and optionally other excipients commonly used in pharmaceutical composition, e.g. oral dosage forms, e.g. tablets.

Various dissolution profiles of different strengths can therefore be obtained either by compressing the same tabletting mixture to tablets of dose proportional weights or by maintaining the same tablet size/weight over all dosage strengths (weight compensation by the excipient used as filler).

Another aspect of the present invention relates to a manufacturing process of the pharmaceutical compositions according to the invention.

The pharmaceutical compositions according to the invention can be prepared by use of well known pharmaceutical processing techniques such as blending, granulation, milling, spray drying, compaction, and coating.
A generic manufacturing procedure of the pharmaceutical composition, e.g. oral dosage forms can be described in the following steps:
Step 1: Place the drug substance, the matrix former(s) (or combinations of them), the binder(s), the disintegrant(s) (if requested), the stabilizer(s) and the filler(s) (if requested, also further components as listed on pages 15-16) into the bowl of the high shear mixer.
Step 2: Mix (e.g., 5 minutes)
Step 3: Add the water solution to the mixture of step (2) (eventually a water soluble stabilizer can be dissolved in the granulating liquid)
Step 4: Mix/knead/granulate the compounds.
Step 5 (optional): Screen the wet granulate (e.g., a sieve of 2 mm mesh size).
Step 6: Dry the granulate on trays or in a fluid bed dryer (preferred).
Step 7: Screen the filler(s), disintegrant(s), glidant(s)/flow agent(s), lubricant(s) and dried granulate into the container of a free fall mixer (e.g., a sieve of 1 mm mesh size).
Step 8: Mix the components of step (7).
Step 9: Compress the tabletting mixture of step (8) on a force feeding (rotary) tabletting machine to tablet cores of the required weight and dimensions.
Step 10: Prepare a suspension of the coloring agent(s), titanium dioxide (white pigment) and talc (glidant) in the required liquid. Add suspending polymer (s) and plasticizer(s), if required.
Step 11: Spray the suspension of step (10) on the cores of step (9) until the required weight of the film coat is achieved.
Step 12: Disperse the enteric polymer(s) in the required liquid (solvents mixture or purified water). Add the talc (glidant) and the polyethylene glycol (plasticizer).
Step 13: Stir the mixture until a homogeneous dispersion/solution is obtained
Step 14: Spray the suspension of step (13) on the film-coated tablets of step (11) until the required weight of the film coat is achieved.
Said process can be generalized as follow:
mixing of the inner phase components comprising the drug substance, the matrix former and the stabiliser
granulation (a water soluble stabilizer can eventually be dissolved in the granulating liquid)
mixing of the granulate with the outer phase components
compression of the tabletting mixture into tablet cores
coating of the tablet cores with the non-functional coat
coating of the film-coated tablets with the enteric coat

Table 4 discloses particle size distribution of the tabletting mixture (including outer phase components).

The particle size distribution of the tabletting mixture is determined with the sieve analysis residue method and can vary in a broad range.

| | Finest tabletting | Coarsest tabletting |
|---|---|---|
| μm | mixture | mixture |
| 0 | 10.7 | 66 |
| 90 | 5.4 | 5.2 |
| 125 | 9.1 | 3.9 |
| 180 | 8.7 | 1.8 |
| 250 | 13.4 | 2.4 |
| 355 | 12 | 2.8 |
| 500 | 19.9 | 5.6 |
| 710 | 20.2 | 11.6 |
| 1000 | 0.5 | 0.8 |

This table can be read as follows:

The 5.4 value means 5.4% in wt of the tabletting mixture consists of particles whose size is between 90 and 125 μm.

In a preferred embodiment of the invention, The table 5 below shows the different ranges of the components of e.g. a pitavastatin sustained release formulation according to the invention, when the matrix forming HPMC components of the internal phase are a combination of a matrix forming HPMC having a viscosity of about 100 000 cps and a matrix forming HPMC having a viscosity of about 100 cps.

All following percentages correspond to percentage by weight of the total core:

TABLE 5
Core

| | |
|---|---|
| NKS 104 (Ca-salt) | From about 1% to 25%, preferably from 1% |
| | to 21%, e.g about 4%, about 8%, about 16%, |
| | about 21% |
| METHOCEL 100T | from about 0 to about 40% (corresponding |
| (100 000 centipoises) | to from about 0 mg to 32 mg) |
| METHOCEL K100 | from about 0 to about 40% (corresponding |
| LVP CR | to from about 0 mg to 32 mg) |
| (100 centipoises) | |
| Avicel PH 101 (filler) | from about 30 to about 80% (corresponding |
| | to from about 24 mg to about 64 mg) |
| Cellulose HP-M 603 | about 5%, e.g. from 2% to 7% |
| (3 cps): binder | |
| Neusilin FH-2 | about 4% e.g. from 2% to 6% |
| Tablet core = 80 mg | |

External phase

| | |
|---|---|
| Magnesium stearate | about 0.5% e.g. from 0.25% to 2% |
| Aerosil 200 | about 0.5% e.g. from 0.1% to 1% |

The following examples are intended to illustrate the invention in various of its embodiments without being limitative in anyway thereof.

### EXAMPLE 1

Core (percentage related to core weight):4.18 mg (5.225% wt) of drug substance, for example pitavastatin Ca-salts, 42.82 mg (53.525% wt) of microcrystalline cellulose, 4 mg (5% wt) of HPMC (3 cps), 25 mg (31.25% wt) of HPMC (100 cps), 3.2 mg (4% wt) of Neusilin, the external phase comprising 0.4 mg (0.5% wt) of silicium dioxide colloidal and 0.4 mg (0.5% wt) of magnesium stearate.

HPMC subcoat (non functional coat) (percentage related to subcoat weight): 2.856 mg (71.4% wt) of Hydroxypropylmethylcellulose 3 cps, 0.286 mg (7.15% wt) of polyethyleneglycol, 0.286 mg (7.15% wt) of talc and 0.572 mg (14.3% wt) of titanium dioxide. Enteric coat (percentage related to enteric coat weight): 5 mg (83.34% wt) of Eudragit L30D, 0.5 mg (8.33% wt) of talc and 0.5 mg (8.33% wt) of polyethyleneglycol.

### EXAMPLE 2

Core (percentage related to core weight): 8.36 mg (10.45% wt) of drug substance, for example pitavastatin Ca-salts, 38.64 mg (48.3% wt) of microcrystalline cellulose, 4 mg (5% wt) of HPMC (3 cps), 25 mg (31.25% wt) of HPMC (100 cps), 3.2 mg (4% wt) of Neusilin, the external phase comprising 0.4 mg (0.5% wt) of silicium dioxide colloidal and 0.4 mg (0.5% wt) of magnesium stearate.

HPMC subcoat (non functional coat) (percentage related to subcoat weight): 2.856 mg (71.4% wt) of Hydroxypropylmethylcellulose 3 cps, 0.286 mg (7.15% wt) of polyethyleneglycol, 0.286 mg (7.15% wt) of talc and 0.572 mg (14.3% wt) of titanium dioxide. Enteric coat (percentage related to enteric coat weight): 5 mg (83.34% wt) of Eudragit L30D, 0.5 mg (8.33% wt) of talc and 0.5 mg (8.33% wt) of polyethyleneglycol.

### EXAMPLE 3

Core (percentage related to core weight):16.72 mg (20.9% wt) of drug substance, for example pitavastatin Ca-salts, 30.28 mg (37.85% wt) of microcrystalline cellulose, 4 mg (5% wt) of HPMC (3 cps), 25 mg (31.25% wt) of HPMC (100 cps), 3.2 mg (4% wt) of Neusilin, the external phase comprising 0.4 mg (0.5% wt) of silicium dioxide colloidal and 0.4 mg (0.5% wt) of magnesium stearate.

HPMC subcoat (non functional coat) (percentage related to subcoat weight): 2.856 mg (71.4% wt) of Hydroxypropylmethylcellulose 3 cps, 0.286 mg (7.15% wt) of polyethyleneglycol, 0.286 mg (7.15% wt) of talc and 0.572 mg (14.3% wt) of titanium dioxide. Enteric coat (percentage related to enteric coat weight): 5 mg (83.34% wt) of Eudragit L30D, 0.5 mg (8.33% wt) of talc and 0.5 mg (8.33% wt) of polyethyleneglycol.

### EXAMPLE 4

Core (percentage related to core weight):3.135 mg (3.92% wt) of drug substance, for example pitavastatin Ca-salts, 43.865 mg (54.83% wt) of microcrystalline cellulose, 4 mg (5% wt) of HPMC (3 cps), 12.50 mg (15.625% wt) of HPMC (100 cps), 12.50 mg (15.625%) of HPMC (100 000 cps), 3.2 mg (4% wt) of Neusilin, the external phase comprising 0.4 mg (0.5% wt) of silicium dioxide colloidal and 0.4 mg (0.5% wt) of magnesium stearate. HPMC subcoat (non functional coat) (percentage related to subcoat weight): 2.856 mg (71.4% wt) of Hydroxypropylmethylcellulose 3 cps, 0.286 mg (7.15% wt) of polyethyleneglycol, 0.286 mg (7.15% wt) of talc and 0.572 mg (14.3% wt) of titanium dioxide. Enteric coat (percentage related to enteric coat weight): 5 mg (83.34% wt) of Eudragit L30D, 0.5 mg (8.33% wt) of talc and 0.5 mg (8.33% wt) of polyethyleneglycol.

### EXAMPLE 5

Core (percentage related to core weight): 6.27 mg (7.84% wt) of drug substance, for example pitavastatin Ca-salts, 40.73 mg (% 50.91 wt) of microcrystalline cellulose, 4 mg (5% wt) of HPMC (3 cps), 16.64 mg (20.8% wt) of HPMC (100 cps), 8.36 mg (10.45%) of HPMC (100 000 cps), 3.2 mg (4% wt) of Neusilin, the external phase comprising 0.4 mg (0.5% wt) of silicium dioxide colloidal and 0.4 mg (0.5% wt) of magnesium stearate. HPMC subcoat (non functional coat) (percentage related to subcoat weight): 2.856 mg (71.4% wt) of Hydroxypropylmethylcellulose 3 cps, 0.286 mg (7.15% wt) of polyethyleneglycol, 0.286 mg (7.15% wt) of talc and 0.572 mg (14.3% wt) of titanium dioxide. Enteric coat (percentage related to enteric coat weight): 5 mg (83.34% wt) of Eudragit L30D, 0.5 mg (8.33% wt) of talc and 0.5 mg (8.33% wt) of polyethyleneglycol.

### EXAMPLE 6

Core (percentage related to core weight):12.54 mg (15.675% wt) of drug substance, for example pitavastatin Ca-salts, 34.46 mg (43.075% wt) of microcrystalline cellulose, 4 mg (5% wt) of HPMC (3 cps), 18.75 mg (23.4375% wt) of HPMC (100 cps), 6.25 mg (7.8125% wt) of HPMC (100 000 cps), 3.2 mg (4% wt) of Neusilin, the external phase comprising 0.4 mg (0.5% wt) of silicium dioxide colloidal and 0.4 mg (0.5% wt) of magnesium stearate. HPMC subcoat (non functional coat) (percentage related to subcoat weight): 2.856 mg (71.4% wt) of Hydroxypropylmethylcellulose 3 cps, 0.286 mg (7.15% wt) of polyethyleneglycol, 0.286 mg (7.15% wt) of talc and 0.572 mg (14.3% wt) of titanium dioxide. Enteric coat (percentage related to enteric coat weight): 5 mg (83.34% wt) of Eudragit L30D, 0.5 mg (8.33% wt) of talc and 0.5 mg (8.33% wt) of polyethyleneglycol.

### EXAMPLE 7

Core (percentage related to core weight): 16.72 mg (20.9% wt) of drug substance, for example pitavastatin Ca-salts, 30.28 mg (37.85% wt) of microcrystalline cellulose, 4 mg (5% wt) of HPMC (3 cps), 20 mg (25% wt) of HPMC (100 cps), 5 mg (6.25% wt) of HPMC (100 000 cps), 3.2 mg (4% wt) of Neusilin, the external phase comprising 0.4 mg (0.5% wt) of silicium dioxide colloidal and 0.4 mg (0.5% wt) of magnesium stearate. HPMC subcoat (non functional coat) (percentage related to subcoat weight): 2.856 mg (71.4% wt) of Hydroxypropylmethylcellulose 3 cps, 0.286 mg (7.15% wt) of polyethyleneglycol, 0.286 mg (7.15% wt) of talc and 0.572 mg (14.3% wt) of titanium dioxide. Enteric coat (percentage related to enteric coat weight): 5 mg (83.34% wt) of Eudragit L30D, 0.5 mg (8.33% wt) of talc and 0.5 mg (8.33% wt) of polyethyleneglycol

The present invention also relates to a pharmaceutical composition as herein above disclosed for sustained release comprising as active ingredient pitavastatin or a pharmaceutically acceptable salt thereof, said composition comprising a core consisting of an inner phase (internal) and an outer phase (external)

wherein the outer phase does not comprise a matrix former and

wherein the core is first coated with a non functional film coat and then with an enteric coat, excluding the following compositions:

a) Inner phase:10.45 wt % of drug substance, for example pitavastatin Ca-salt, 44.8 wt % of microcrystalline cellulose, 5 wt % of HPMC (3 cps), 37.5 wt % HPMC (100 cps), 1.25 wt % of inorganic water-soluble compound (such as potassium bicarbonate) or water insoluble compound (such as neusilin), the external phase comprising 0.5 wt % of silicium dioxide colloidal and 0.5 wt % of magnesium stearate.

b) Inner phase:10.45 wt % of drug substance, for example pitavastatin Ca-salt, 51.05 wt % of microcrystalline cellulose, 5 wt % of HPMC (3 cps), 31.25 wt % HPMC (100 cps) 1.25 wt % of inorganic water-soluble compound (such as potassium bicarbonate) or water insoluble compound (such as neusilin), the external phase comprising 0.5 wt % of silicium dioxide colloidal and 0.5 wt % of magnesium stearate.

The present invention also relates to a pharmaceutical composition for the treatment of hyperlipidemia, hypercholesterolemia and atherosclerosis, as well as other diseases or conditions in which HMG-CoA reductase is implicated comprising an HMG-CoA reductase inhibitor or a pharmaceutically acceptable salt thereof, said composition comprising a core consisting of an inner phase (internal) and an outer phase (external) wherein the outer phase does not comprise a matrix former and

wherein the core is first coated with a non functional film coat and then with an enteric coat.

The present invention also relates to a method of treatment of hyperlipidemia, hypercholesterolemia and atherosclerosis, as well as other diseases or conditions in which HMG-CoA reductase is implicated comprising administering to a patient in need thereof a therapeutically effective amount of a composition according to the invention.

The present invention also concerns the use of the composition according to the invention in the manufacture of a medicament for use in the treatment or prevention of a cardiovascular disease, e.g., hypercholesterolemia, hyperproteinemia and/or atherosclerosis.

In a preferred embodiment the invention relates to the use of the composition according to the invention in the manufacture of a medicament wherein said medicament is a hyperlipidemic, hypercholesteremic, hyperlipoproteinemic or anti-atherosclerotic agent.

1. A pharmaceutical composition for sustained release, comprising:
as active ingredient pitavastatin or a pharmaceutically acceptable salt thereof, wherein said composition comprising a core consisting of an inner phase (internal) and an outer phase (external) wherein the outer phase does not comprise a matrix former and wherein the core is first coated with a non functional film coat and then with an enteric coat.

2. The composition according to claim 1, wherein the amount of pitavastatin or pharmaceutically acceptable salt thereof is about 1-50 weight % of the core composition.

3. The composition according to claim 2, wherein the amount of pitavastatin or pharmaceutically acceptable salt thereof is about 5-50 weight % of the core composition.

4. The composition according to claim 1, wherein the amount of pitavastatin or pharmaceutically acceptable salt thereof is about 1-32 mg.

5. The composition according to claim 1, wherein the inner phase comprises a matrix former.

6. The composition according to claim 5, wherein the matrix former comprises one or more types of matrix former component having different viscosities.

7. The composition according to claim 5, wherein the matrix former is selected from the group consisting of polyethylene glycol, polyvinylpyrrolidone, polyvinyl alcohol, hydrophilic polymers such as hydroxypropylcellulose, hydroxymethylcellulose, and hydroxypropylmethylcellulose.

8. The composition according to claim 7, wherein the matrix former is hydroxypropylmethylcellulose (HPMC).

9. The composition according to claim 8 wherein the amount of HPMC as a matrix former is about 1-60 weight % (based on the total core components).

10. The composition according to claim 9, wherein the matrix former of the inner phase has a viscosity of about 1 to about 100.000 cps.

11. The composition according to claim 9, wherein the matrix former of the inner phase has a viscosity of about 1 to about 500 cps.

12. The composition according to claim 1, wherein said composition comprises a stabilizer.

13. The composition according to claim 12, wherein the stabilizer is magnesium aluminometasilicate (neusilin).

14. The composition according to claim 13, wherein the amount of the stabilizer is about 1-15 weight % (based on the total core components).

15. The composition according to claim 1, wherein the non-functional coat consists in Hydroxypropylmethylcelluloce, Polyethyleneglycol, titanium dioxide and talc.

16. The composition according to claim 1, wherein the amount of non functional film coat is used at about 4 mg of film coat pro cm².

17. The composition according to claim 1, wherein the enteric coat consists of methacrylic copolymer, talc and polyethyleneglycol.

18. The composition according to claim 1, wherein the enteric coat is used at 4 to 6 mg polymer pro cm².

19. A method of treatment of hyperlipidemia, hypercholesterolemia and atherosclerosis, as well as other diseases or conditions in which HMG-CoA reductase is implicated, comprising:
administering to a patient in need thereof a therapeutically effective amount of a composition according to claim 1.

20. (canceled)
